(19) United States
(12) Patent Application Publication
(10) Pub. No.: US 2022/0125984 A1
(43) Pub. Date: Apr. 28, 2022

(54) System and Method for Odor Treatment

(71) Applicant: Regina Crisci, Winterville, NC (US)

(72) Inventor: Regina Crisci, Winterville, NC (US)

(21) Appl. No.: 17/082,276

(22) Filed: Oct. 28, 2020

(51) Int. Cl. A61L 9/014; B65D 43/02; B65D 47/04

(52) U.S. Cl. CPC A61L 9/014; B65D 2251/20; B65D 47/046; B65D 43/0235

(57) ABSTRACT

The present invention relates to a system and method for odor treatment. In most embodiments the system comprises a container comprising a receptacle and a lid which is filled with a mixture of odor treating materials. In many embodiments this odor treating material mixture is a combination of sodium bicarbonate and activated carbon. In most embodiments the method for odor treatment comprises five main steps. First, the odor treating material is prepared. Second, the odor treating material is dispensed or placed within the container. Third, the container is sealed, closed, or covered. Fourth, the container is opened. And fifth, the odor treating material is dispensed from container directly onto an odor causing material.

### BACKGROUND OF THE INVENTION

The present invention relates to a system and method for odor treatment.

Often times individuals come in contact with bad smelling or odiferous objects in their daily life. Things such as old food, garbage, compost bins, used diapers, body odor, or excrement, are common items, but can be unpleasant to deal with and can make the home or work environment disagreeable. Many individuals will try to mask the smell with deodorants, perfumes, air fresheners, or disinfectant sprays. Often, however, these only cover up the smell and do not deal with the problem itself. Additionally, many times instead of masking, these proposed solutions simply mix with the odor and cause an unpleasant smell consisting of artificial scents and odors. This mixture can be especially disagreeable to those with allergies to perfumes, those with sensitive olfactory senses, or to those who get migraines with heavy chemical use.

A common odiferous object, especially for parents with small children or caretakers of older or handicap individuals, is used diapers. Often used diapers are kept in special containers which are designed to keep odors inside of the container, but often the containers are not fully air tight and thus release odors over time. Further, typically every time these containers are opened when a new used diaper needs to be discarded, the build up odors are released. Many individuals thus try to open and shut the containers as quickly as possible, or as few times as possible, which rarely works. Others try to roll up the diaper as tightly as possible as a means of trapping the odiferous excrement inside the absorbent diaper layers. Other users simply empty the diaper containers frequently, which is time consuming and wasteful if a liner is used.

As discussed above, some have tried to mask the diaper odors with perfumes or other sprays, and other have tried to sprinkle absorbing materials around or on top of the diaper. However, these methods often do not work as they do not neutralize the odor at its source, namely the excrement found on the used diaper surface.

Thus, what is needed is a system and method for reducing odors. This system should ideally neutralize or absorb the odor rather than just masking it or covering it up. Similarly, the method ideally should apply the system directly to the odor causing object as opposed to the air or other things around the object.

### SUMMARY OF THE INVENTION

The present invention relates to a system and method for odor treatment.

In some embodiments the method for odor treatment comprises the steps of first, preparing an odor treating material, second, providing a container, third, dispensing the odor treating material into the container, fourth, sealing the container with the odor treating material inside of the container, and fifth, dispensing the odor treating material onto an odor causing material. In some embodiments the second step further comprises the steps of providing a receptacle and providing a lid for the receptacle. In some embodiments the fourth step further comprises the steps of securing a lid to the receptacle and closing the lid. In some embodiments the lid is closed after use and stored for later use.

In some embodiments the lid further comprises at least one hole out of which the odor neutralizing material is dispensed. In some embodiments the lid comprises an open and closed position. In some embodiments the lid is removably attachable.

In some embodiments the odor causing material comprises two materials which are mixed together. In some of these embodiments with two materials, the two materials are activated carbon and sodium bicarbonate, which are mixed together in a 10-20% and 80-90% ratio, respectively.

In some embodiments a safety seal is added onto the receptacle, and in other embodiments a safety seal is added over the lid.

In some embodiments, when the odor treating material is dispensed from the container onto the odor causing material, the lid is about 3 inches or less from the odor causing material in order to control particulates.

In some embodiments the method for odor treatment further comprises the steps of first, providing a refill pack, second, dispensing the odor treating material into the refill pack, third, sealing the refill pack, and fourth, dispensing the odor treating material from the refill pack into the container.

In some embodiments the system for odor treatment comprises the odor treating material, a container comprising a receptacle, and a removably attachable lid, which is removably attached to the receptacle by mating threads on the receptacle and lid. In most embodiments the lid further comprises an open and a closed position, and a base and a cover which each comprise at least one hole. In these embodiments, the open position is when the at least one hole in the cover is generally aligned with the at least one hole in the base and the closed position is when the holes are not generally aligned.

In some embodiments the odor causing material comprises two materials which are mixed together. In some of these embodiments with two materials, the two materials are activated carbon and sodium bicarbonate, which are mixed together in a 10-20% and 80-90% ratio, respectively.

In some embodiments multiple containers are used which are different from one another. In some embodiments each container is different in size, in others each container is different in indicia, and in other each container is different due to its odor neutralizing material.

In some embodiments at least two refill packs are included with the odor treating system. In some the multiple refill packs are different from one another. In some embodiments each refill pack is different in size, in others each refill pack is different in indicia, and in other each refill pack is different due to its odor neutralizing material.

### BRIEF DESCRIPTION OF THE DRAWINGS

FIG. 1 is a flowchart describing the general steps of the method for using a system for odor treatment.

FIG. 2 is a flowchart describing the general steps for refilling the system for odor treatment.

FIG. 3 a system for odor treatment according the claimed invention.

FIG. 4 shows an exploded view of the system for odor treatment shown in FIG. 3.

FIG. 5A shows a lid for the system for odor treatment shown in FIG. 3.

FIG. 5B shows a lid for the system for odor treatment shown in FIG. 3.

FIG. 5C shows a lid for the system for odor treatment shown in FIG. 3.

FIG. 6 shows the system for odor treatment as shown in FIG. 3 in the process of being used as described in FIG. 1.

FIG. 7 shows the system for odor treatment as shown in FIG. 3 in the process of being refilled as described in FIG. 2.

FIG. 8 shows a kit made up of different systems for odor treatment.

### DETAILED DESCRIPTION OF THE INVENTION

The present invention relates to a system and method for odor treatment. While this invention disclosure will focus on reducing odors in diapers, it is intended to be used on any odor causing item, as will be discussed in more detail below.

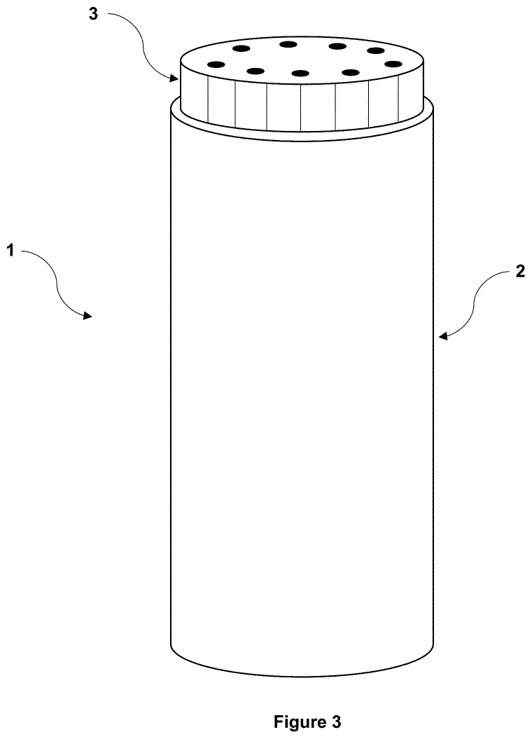

In most embodiments, the system for odor reduction comprises odor treating or absorbing materials (5) placed in a container (1) as shown in FIG. 3. In some embodiments only one material is used and in other embodiments a combination of materials are used. In most embodiments with a combination of materials the combination consists of activated carbon (or activated charcoal) and sodium bicarbonate. In some embodiments sodium carbonate is used instead of sodium bicarbonate. In other embodiments a mixture of sodium carbonate and sodium bicarbonate are used. While activated carbon was found to be a powerful odor neutralizer, as a fine powder, activated carbon has a propensity form particulates, and due to the dark nature of the substance, these particulates can cause difficult to clean messes. Mixing the activated carbon with the sodium bicarbonate was found to beneficially mange these activated carbon particulates.

In some embodiments 10-20% of the mixture is activated carbon and 80-90% of the mixture is sodium bicarbonate. This range was found to be ideal for balancing odor treatment while also limiting the particulates or atmospheric aerosol particles of activated carbon. In other embodiments less than 10% activated carbon is used. These embodiments are useful for odors which do not need as strong of odor treatment, but where the activated carbon particulates need to be highly managed. In other embodiments more than 20% of activated carbon is used. These embodiments are useful for when strong odor treatment is required and the activated carbon particulates are less of a concern.

In some embodiments, other agents are used. In some embodiments these other agents are used in the mixture of sodium bicarbonate and activated carbon, in other embodiments the other agents are used with just activated carbon, in other embodiments the other agents are used with just sodium bicarbonate, and in other embodiments the other agents are used by themselves. The other agents are selected from the group consisting of odor treating beads, fragrant gel beads, perfumes, cologne, deodorants, air fresheners, potpourri, disinfectants, and combinations thereof. In some embodiments these other agents aid in the odor treatment, such as embodiments which use odor treating beads. In other embodiments the other agents complement the odor treatment by also adding a fresh or clean sent to the air, such that the odor is treated and a fresh sent is produced by using the product.

In some embodiments where the other agents are used in combination with sodium bicarbonate or activated charcoal, the other agents make up less than 10% of the total mixture. These embodiments are useful for agents with strong scents or when only a small amount of odor treatment or freshener is needed. In other embodiments the other agents comprise 10-50% of the mixture. These embodiments are useful when the other agent is designed to complement the odor treatment of the activated charcoal or sodium bicarbonate.

In some embodiments the mixtures are percent by weight and in others they are percent by volume.

In some embodiments, the odor treating materials and other agents discussed above are used in a dried powder form, in other embodiments they are prepared as a paste or cream, and in others they are dissolved or suspended in a liquid. Powdered embodiments are useful as they can easily cover large surface areas and do not need to be manually spread or absorbed, but have the downside that they can cause particulates which may be undesirable. Embodiments where the materials are prepared as a paste or cream are useful as they trap the particulates and do not need to be absorbed like a liquid, but have the downside of needing to be spread manually. Embodiments where the materials are dissolved or suspended in a liquid are useful as they can be easily spread without particulates, but have the downside of needing to be absorbed.

While the embodiments above describe using one or more materials, even some which may not neutralize odors but rather act as a deodorant, the material or combination of materials used will be called odor treating material 5 throughout the specification.

In most embodiments odor treating material 5 is placed in container 1. In most embodiments container 1 comprises receptacle 2 and lid 3. In some embodiments no lid 3 is provided, and receptacle 2 comprises the entirety of container 1. In most embodiments lid 3 covers hole 4 in receptacle 2. In most embodiments odor treating material 5 is placed inside and dispensed out of receptacle 2 by hole 4. In some embodiments lid 3 screws onto receptacle 2, in other embodiments it snaps onto receptacle 2, and in others it stops hole 4. In some embodiments lid 3 is removed before dispensing odor treating material 5, and in other embodiments odor treating material 5 is dispensed through lid 3.

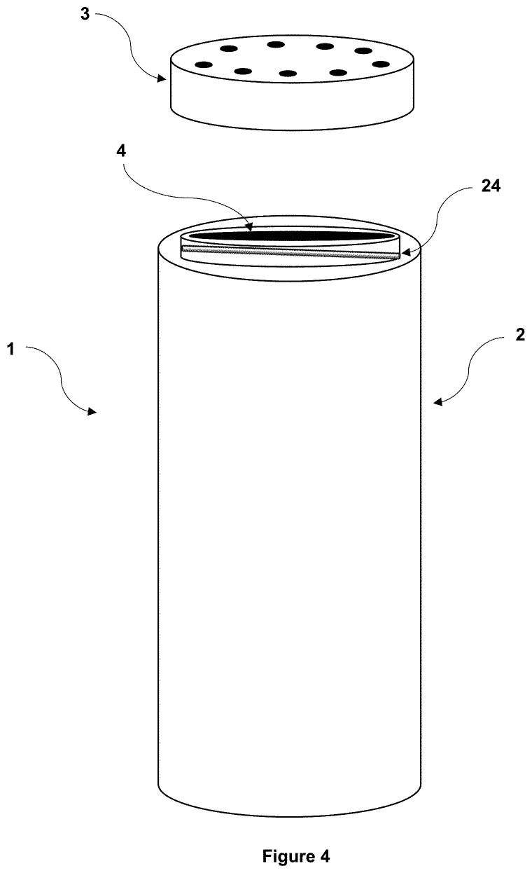

In some embodiments receptacle 2 is a bottle, can, or container, as shown in FIGS. 3 and 4, in other embodiments receptacle 2 is a bag or pouch. In some embodiments receptacle 2 is made of stiff materials selected from the group consisting of wood, metal, plastic, paper, glass, rubber, and combinations thereof. In other embodiments receptacle 2 is made of malleable materials selected from the group consisting of plastic, paper, rubber, and combinations thereof. Embodiments where receptacle 2 is made of a stiff material are useful when odor treating material 5 is poured or sprinkled out of the container, or when a collapsing of the container would cause fine particles to be rapidly released in an undesirable way. Embodiments where receptacle 2 is made of a flexible material is useful when receptacle 2 is squeezed or collapsed to force out odor treating material 5, such as embodiments where odor treating material 5 is dissolved or suspended in a liquid.

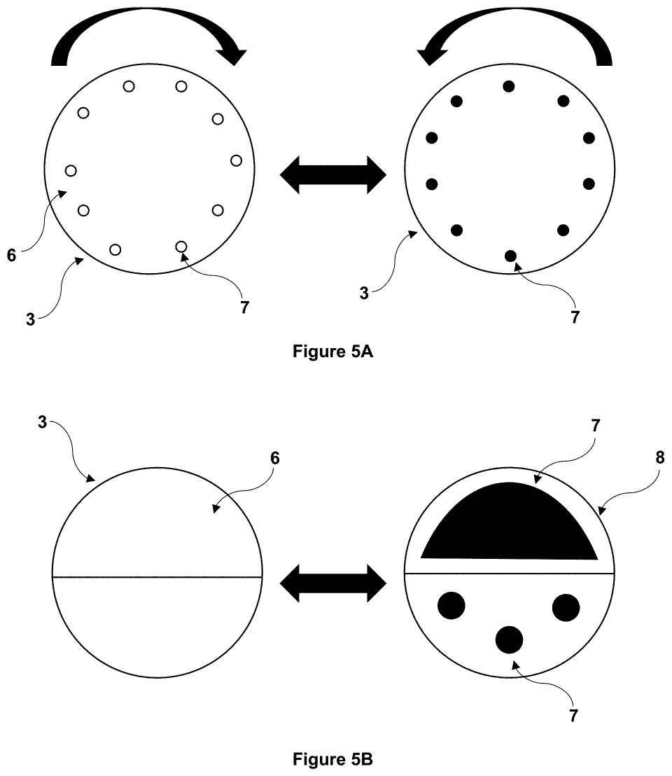

FIGS. 5A, 5B, and 5C show different types of lid 3 embodiments where odor treating material 5 would be dispensed through.

FIG. 5A shows a first embodiment where lid 3 rotates to open and close. In these embodiments lid 3 comprises cover 6 and base 8. Base 8 engages with receptacle 2 in the methods described above (namely screwing, snapping, stopping) and cover 6 rotates around base 8 of lid 3. Both cover 6 and base 8 comprise holes 7 where odor treating material 5 is dispensed out of. The left side of FIG. 5A shows lid 3 in the closed position, where holes 7 in cover 6 are not aligned with holes 7 on base 8. The right side of FIG. 5A shows lid 3 in the open position, where holes 7 in cover 6 are aligned with holes 7 on base 8. As shown by the arrow above the lids, cover 6 is rotated in a clockwise or counterclockwise direction to align or un-align holes 7 and thus put lid 3 in an open or closed position, as described.

FIG. 5B shows a second lid 3 embodiment where cover 6 is secured to base 8 and is flipped open and closed. In some embodiments, such as the one shown in FIG. 5B, cover 6 is split into sections which can each be individually opened. These embodiments are useful when the different sections have different sized holes 7, as seen in the top and bottom holes 7 on the right side of FIG. 5B. These embodiments are also useful even if all of the holes 7 are the same size as it allows the user to control the number of open holes 7 and thus the amount of odor treating material 5 which is able to be dispensed out of holes 7 at a given time. In other embodiments, the entire cover 6 is removed as a single piece. In some embodiments, at least a portion of cover 6 is secured to the base. These embodiments are useful as it prevents cover 6 from being misplaced or lost once removed. In other embodiments cover 6 is completely removed. These embodiments are useful as they avoid the potential for cover 6 to interfere with the dispensing of odor treating material 5 or accidentally hitting or engaging with odor causing material 11 onto which odor treating material 5 is being dispensed. In other embodiments cover 6 is secured with tether 10, as shown in FIG. 5C.

FIG. 5C shows a third embodiments of lid 3, which comprises base 8, funnel 9 and cover 6. As described above, in most embodiments base 8 engages with receptacle 2 in the methods described above (namely screwing, snapping, stopping). Funnel 9 beneficially funnels odor treating material 5 to a single location and allows for more precise and targeted dispensing. Cover 6 covers funnel 9. In some embodiments cover 6 is a cap which is secured to funnel 9 and in other embodiments it is a stopper which stops funnel 9. As described above, in some embodiments cover 6 is partially secured to funnel 9, in other embodiments it is fully removed when not covering funnel 9, and in others cover 6 is secured by tether 10. In some embodiments tether 10 tethers base 8 and cover 6 and in others it tethers cover 6 and funnel 9. Funnel 9 is especially useful for dispensing fine powders or liquids. In some embodiments funnel 9 is fully open on its dispensing end, and in others funnel 9 is further reduced with holes 7.

In some embodiments holes 7 are between 0.5-2 millimeters (mm) in diameter. These embodiments are useful for when odor treating material 5 is a fine powder whose particulates need to be highly controlled. In other embodiments holes 7 are between 2-5 mm in diameter. These embodiments are useful when odor treating material 5 is less likely to form particulates or when more material is desired to be dispensed with each inversion or shaking of container 1. In other embodiments holes 7 are between 5-9 mm in diameter. These embodiments are useful when odor treating material 5 is bulkier materials such as the odor treating beads. In other embodiments holes 7 are greater than 9 mm in diameter. These embodiments are useful when significant amounts of odor treating material 5 is needed or when odor treating material 5 comprises larger pieces such a potpourri. As discussed above, in some embodiments lid 3 comprises holes 7 of all the same size, and in others there are different hole 7 sizes.

In many embodiments the size of hole 7 correlates with the number of holes 7 on lid 3. In other embodiments the diameter of lid 3 correlates with the number of holes 7, with larger lids comprising more holes 7 than smaller lids. In some embodiments there is a single hole 7 on lid 3. These embodiments are useful in combination with large hole 7 sizes. This embodiment would also describe embodiments with a funnel 9 and no holes 7. In other embodiments there are 2-5 holes 7 on lid 3. These embodiments are useful with larger hole 7 sizes or on lids with smaller diameters. In some embodiments there are 5-9 holes 7 on lid 3. These embodiments are useful with smaller hole 7 sizes. In other embodiments there are more than 9 holes 7. These embodiments are useful with smaller hole 7 sizes or with larger lid 3 diameters. In other embodiments there are more than 9 holes 7.

In some embodiments lid 3 is a septum or similar object which can be pierced by a needle. In some embodiments lid 3 is equipped with child-proof features which limit younger children from opening receptacle 2. These embodiments are important as in some embodiments the contents of receptacle 2 are messy or hazardous and thus should not be accessed and used by small children.

In some embodiments lid 3 is removably attached to receptacle 2. These embodiments are useful as they allow lid 3 to be removed and receptacle 2 to be refilled. Removably attachable lid 3 is attached to receptacle 2 by the methods consisting of engaging threads which screw into one another, mating ridges which snap into one another, and combinations thereof. In other embodiments, lid 3 is integrally connected to receptacle 2 and is not designed to be removed. These embodiments are useful when container 1 is disposable such that when it is empty the entire container 1 is disposed or recycled.

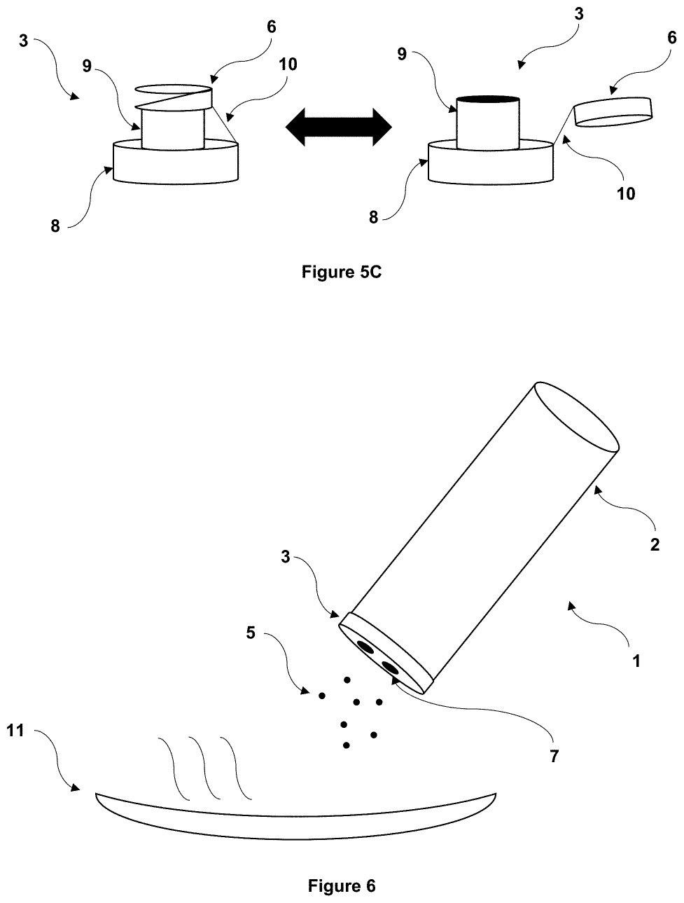

FIG. 6 shows container 1 in use, dispensing odor treating material 5 onto the odor causing material 11. As shown and previously described, receptacle 2 holds odor treating material 5 until inverted or in some way dispensed out of receptacle 2. In the embodiment shown, receptacle 2 is inverted which dispenses odor treating material 5 out of holes 7 on lid 3 and onto odor causing material 11.

In some embodiments container 1 comprises only a receptacle 2. In some embodiments receptacle 2 is resealable and in others once receptacle 2 is opened it must be sealed or closed by another method. In some embodiments the resealable mechanism is a zipper, in others embodiments it is an interlocking mechanism, and in others is it a hook and pile system.

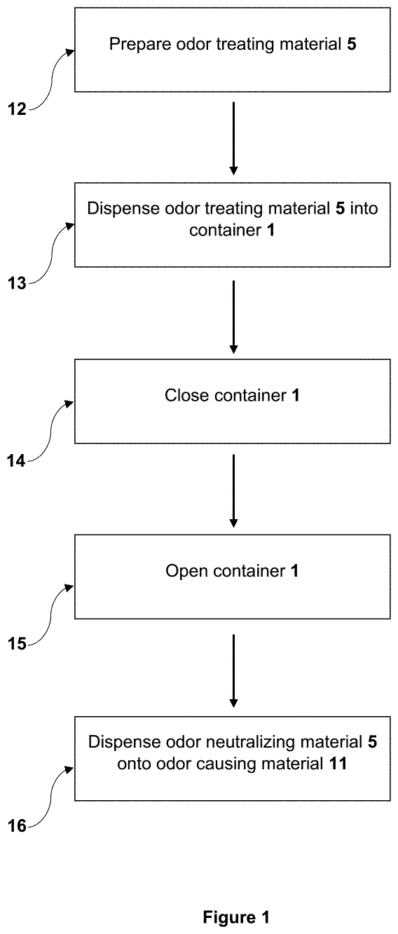

With this description of the odor treating system, the method of using this system will now be discussed. As shown in FIG. 1, the method for odor treatment comprises five main steps. First, (12) odor treating material 5 is prepared, second, (13) odor treating material 5 is dispensed or placed within container 1, third, (14) container 1 is sealed, closed, or covered, fourth, (15) container 1 is opened and fifth (16) odor treating material 5 is dispensed from container 1. Each of these steps will be discussed in more detail below.

The first step (12) involves preparing odor treating material 5. This preparation is dependent on the nature and formulation of odor treating material 5. For all embodiments the components of odor treating material 5 must be purchased or created. For embodiments where odor treating material 5 is a mixture of powders, step 12 involves mixing or otherwise blending the powdered materials together to form a generally evenly distributed mixture. For embodiments where odor treating material 5 is a cream or paste the particulate matter is suspended and mixed in the cream, fat, emulsion, or other material to form a generally evenly distributed mixture. For embodiments where odor treating material 5 is suspended or dissolved in a liquid, the powdered substance is added to the liquid, or the liquid added to the powdered substance, and mixed to form a generally evenly distributed mixture.

The second step (13) involves dispensing odor treating material 5 into container 1. The dispensing of odor treating material 5 in most embodiments depends on the size and shape of container 1 and hole 4, as well as the formulation of odor treating material 5. In most embodiments the dispensing method is selected from the group consisting of pouring, injecting, funneling, sifting, placing, and combinations thereof. In some embodiments the entire mixture made in step 12 is dispensed into container 1 and in others it is dispensed into multiple containers.

The third step (14) involves sealing, closing, or covering container 1. In embodiments where lid 3 is removably affixed to receptacle 2, this step involves fixing lid 3 to receptacle 2, which involves screwing, snapping, or otherwise securing lid 3 to receptacle 2. In some embodiments where lid 3 is integrally connected to receptacle 2 step 14 involves fusing or otherwise integrally connecting lid 3. In some embodiments where lid 3 is integrally connected to receptacle 2, step 14 involves closing lid 3. In other embodiments, receptacle 2 is sealed without a lid 3. These embodiments are useful when receptacle 2 is a malleable material and which hold odor treating material 5 in a sealed bag. In some embodiments receptacle 2 is fused together, in others receptacle 2 is stapled together, in others receptacle 2 is glued together, and in others it is molded together. In some of these embodiments with no lid 3, receptacle 2 is equipped with a sealing mechanism as discussed above, and the sealing mechanism is secured together to close. As discussed above, in some embodiments a septum or other sealing unit is used as a lid 3. In some of these embodiments step 14 involves securing the septum to receptacle 2 and in others no sealing step is necessary as the septum is already sealing the contents inside of receptacle 2.

In some embodiments hole 4 is further equipped with a safety seal which is removed before the first use. In some embodiments lid 3 is further equipped with a safety seal which is removed before the first use. In some embodiments both hole 4 and lid 3 are equipped with a safety seal which is removed before the first use.

The fourth step (15) involves opening container 1. In most embodiments opening container 1 involves opening lid 3, as discussed above. In embodiments without a lid 3, opening container 1 involves breaking the sealed receptacle 2, in others it involves opening the resealable seal, in others it involves piercing the septum.

The fifth step (16) involves dispensing odor treating material 5. In most embodiments, odor treating material 5 is dispensed onto odor causing material 11, as seen in FIG. 6. In most embodiments, odor treating material 5 is dispensed out of receptacle 2 as it was dispensed into receptacle 2, namely pouring, injecting, funneling, sifting, placing, and combinations thereof. In most embodiments where odor treating material 5 is a powder or dissolved in a liquid, odor treating material 5 is poured or sifted out of container 1.

In most embodiments the pouring action is completed less than 1 inch from odor causing material 11. This method causes the least particulates or liquid splashes from forming between container 1 and odor causing material 11, but runs the risk of container 1 itself engaging with odor causing material 11. In other embodiments the pouring action is completed between 1-3 inches from odor causing material 11. This range beneficially limits particulates or liquid splashed as well as limiting the direct engagement of container 1 with odor causing material 11. In other embodiments, the pouring action is completed over 3 inches from odor causing material 11 which beneficially gives the least possibility of direct contact between container 1 and odor causing material 11.

In most embodiments where odor treating material 5 is suspended in a cream, odor treating material 5 is applied or rubbed directly onto odor causing material 11.

In most embodiments, less than 40% of odor causing material 11 is covered by or engaged with odor treating material 5. These embodiments are useful for mild odors or when the conservation of odor treating material 5 is desired. In other embodiments 40-70% of odor causing material 11 is covered by or engaged with odor treating material 5. These embodiments beneficially treat a majority of odor causing material 11 while conserving odor treating material 5. In other embodiments over 70% of odor causing material 11 is covered by or engaged with odor treating material 5. In other embodiments 100% of odor causing material 11 is covered by or engaged with odor treating material 5. These embodiments beneficially maximize the odor reduction and are useful for powerful or extremely unpleasant odors.

In embodiments where container 1 can be resealed, step 16 has an optional step of resealing container 1 and storing container 1 for future use, wherein step 16 would be repeated until receptacle 2 is empty.

In most embodiments odor treating material 5 is applied directly to odor causing material 11. These embodiments beneficially allow odor treating material to directly engage with and treat the odor at its source. In other embodiments odor treating material 5 is applied around odor causing material 11 but not directly engaging with it. These embodiments can still beneficially neutralize the odors coming off of odor causing material 11, but will limit the ability to neutralize the source of the odor as opposed to its effects. In other embodiments odor treating material 5 is dispensed in an area that is not currently engaging with odor causing material but which is folded onto or later causes odor treating material 5 to odor causing material 11. An example of this would be applying odor treating material 5 onto the top end of a used diaper, away from the human excrement, but then folding the top of the diaper onto the bottom such that odor treating material 5 folds onto and engages with the odor causing human excrement. This method beneficially limits the potential for container 1 to engage with odor causing material 11 while also maximizing the odor treating ability of odor treating material 5.

In most embodiments, once applied, odor treating material 5 is left engaging with odor causing material 11 and in other embodiments odor treating material 5 is removed from odor causing material 11 after a period of time.

An example will be given of this system and method to demonstrate a single embodiment. This example is not intended to be limiting, but to show one embodiment of how the above-described invention would be manufactured and used.

For this example, the system is a powdered mixture of 20% by weight of activated carbon and 80% by weight of sodium bicarbonate in a receptacle 2 with a rotating lid 3 which is removably attached to receptacle 2 by mating threads 24 which screw into one another. Lid 3 comprises 9 holes whose diameter is 1.5 mm each. Odor causing material 11 is a used diaper.

The method is as follows. First (step 12), 200 grams of activated carbon and 800 grams of sodium bicarbonate are mixed together to form 1000 grams of a generally evenly distributed odor treating material 5 powder comprising 20% by weight activated carbon and 80% by weight sodium bicarbonate. Second (step 13), 100 grams of this odor treating material 5 is poured into 10 different receptacles 2 with a funnel. Third (step 14), for each container 1, a safety seal is placed over hole 4, and a lid 3 is screwed onto receptacle 2. Lid 3 is screwed on in the closed position. Fourth (step 15), the user would unscrew lid 3 from receptacle 2 and remove the safety seal over hole 4. Then the user would re-screw lid 3 onto receptacle 2 and rotate open lid 3. Fifth (step 16), after removing the diaper, odor treating material 5 is applied to the inside of the diaper, directly on the human waste and is dispensed about 2 inches from the odor causing human waste. The diaper is then rolled up and discarded as usual, allowing odor treating material 5 to continually engage with the human waste. Finally, lid 3 is rotated shut and stored for the next diaper change.

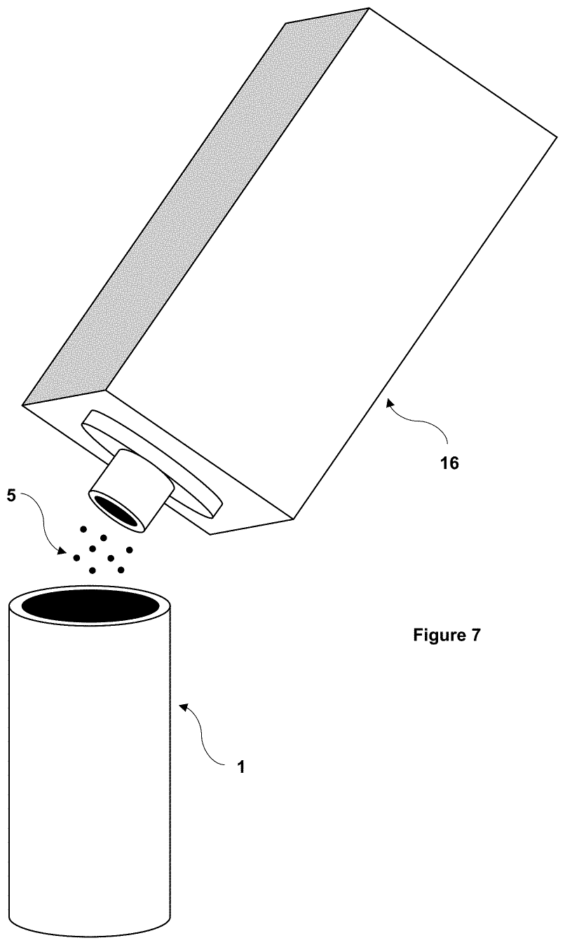

In some embodiments refill pack 17 is provided which allows a user to refill their container 1, as shown in FIG. 7. In most embodiments the composition of odor treating material 5 in refill pack 17 is essentially the same as the composition was in original container 1 and in other embodiments the composition is different. Compositions which are essentially the same allow the user to know that the composition will work similarly to the odor treating material 5 they have been using, and compositions which are different allow a user to try different compositions or for a manufacturer to modify the composition if better ratios, materials, or other composition factors are discovered. In most embodiments the description of container 1 above also describes the container of refill pack 17. In some embodiments refill pack 17 is approximately the same size as container 1 such that a single refill pack 17 refills a single container 1. In most of these embodiments the container of refill pack 17 is a single use disposable container. In other embodiments refill pack 17 is larger than container 1 such that a single refill pack 17 refills multiple containers 1. In most of these embodiments refill pack 17 is resealable or able to be opened and closed.

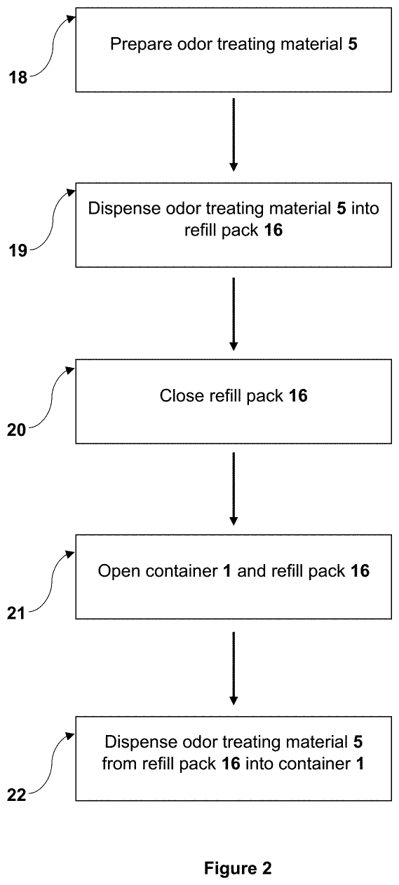

FIG. 2 shows a general method for preparing and using refill pack 17, which is similar to the method shown in FIG. 1. First (step 18), odor treating material 5 is prepared as in step 12. Second (step 19), odor treating material 5 is dispensed into refill pack 17 as in step 13. Third (step 20), refill pack 17 is sealed or closed as in step 14. Fourth step 21), refill pack 17 is opened and fifth (step 22), odor treating material 5 is dispensed into the original container 1.

In some embodiments, users may use refill pack 17 as a large container 1 as opposed to using a smaller container 1.

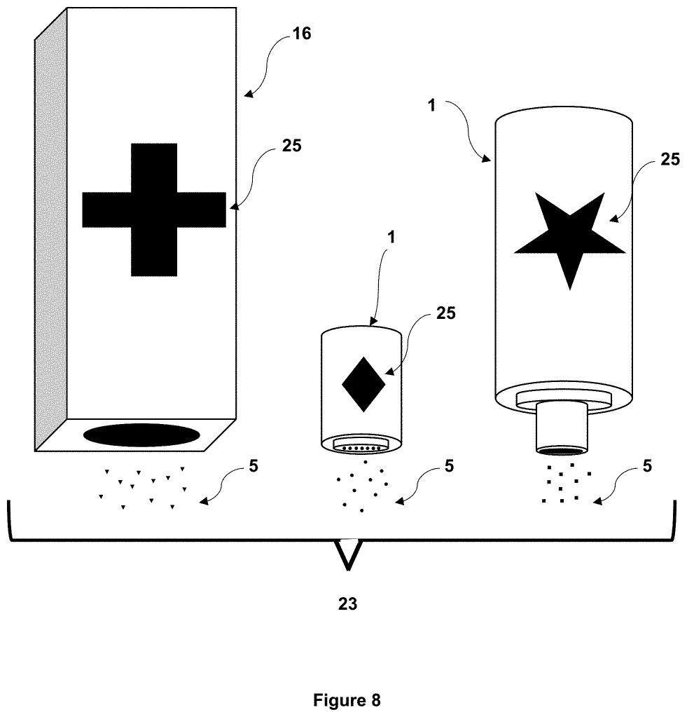

As shown in FIG. 8, in some embodiments the system for odor treatment is kit 23 comprising one or more containers 1 or refill packs 17. In some embodiments kit 23 comprises multiple containers 1 and no refill packs 17, in others kit 23 comprises multiple refill packs 17 and no containers 1, and in others kit 23 comprises at least one container 1 and at least one refill pack 17. In some embodiments the different receptacles are the same size and odor treating material 5 is generally the same composition in all receptacles.

In some embodiments that use kit 23, the different receptacles are different sizes. An example of this would be an embodiment which comprises a larger container 1 which is intended to be used at home where ample shelf space is available and a smaller travel sized container 1 which is intended to be packed in a suitcase, diaper bag, or other bag. These different sized containers can be seen in FIG. 8.

In other embodiments that use kit 23, the different receptacles comprise different odor treating materials 5. As discussed above, odor treating material 5 comprises different compositions in different embodiments, and these different compositions have different advantages and disadvantages depending on the situation. In one kit embodiment there is a container 1 comprising a powdered mixture of 20% by weight of activated carbon and 80% by weight of sodium bicarbonate for odor treatment with no perfume or other scents added and a second container 1 comprising powdered mixture of 18% by weight of activated carbon, 77% by weight of sodium bicarbonate, and 5% by weight fragrant gel beads. This embodiment beneficially gives the user the ability to choose if they want to use the fragrant or non-fragrant variety depending on the situation. These different odor treating material 5 compositions are shown by the different shapes coming from the different containers in FIG. 8.

In some embodiments container 1 and refill pack 16 have different indices 25 which in different embodiments accomplish different purposes. In some embodiments the different indices 25 denote different bottle sizes. In some embodiments the different indices 25 denote different odor treating material 5 compositions. In some embodiments the different indices 25 are designed to be appealing to the user of the receptacle. Indices 25 are selected from the group consisting of color, shape, picture, pattern, or combinations thereof.

While the present invention has been particularly described, it is evident that many alternatives, modifications and variations will be apparent to those skilled in the art in light of the foregoing description. These descriptions and drawings are exemplary of specific embodiments only and are not intended to be limiting to the scope of the invention defined in the claims. It is therefore contemplated that the claims will embrace any such alternatives, modifications, and variations as falling within the true scope and spirit of the present invention.

What is claimed:

1. A method for odor treatment comprising the steps of:
preparing an odor treating material;
providing a container;
dispensing said odor treating material into said container;
sealing said container with said odor treating material inside of said container;
dispensing said odor treating material onto an odor causing material.

2. The method for odor treatment according to claim 1, wherein said odor treating material is prepared by mixing together a first and second material.

3. The method for odor treatment according to claim 2, wherein said odor treating material comprises 10-20% activated carbon and 80-90% sodium bicarbonate.

4. The method for odor treatment according to claim 1, wherein said step of providing a container further comprises the steps of:
providing a receptacle;
providing a lid.

5. The method for odor treatment according to claim 4, wherein said lid is further provided with at least one hole.

6. The method for odor treatment according to claim 4, wherein said lid further comprises an opened and closed position.

7. The method for odor treatment according to claim 7, wherein said step of sealing said container further comprises the steps of:
securing said lid to said receptacle;
closing said lid.

8. A method for odor treatment comprising the steps of:
mixing together at least a first and second material to form an odor treating material;
providing a container comprising a receptacle and a removably attachable lid;
dispensing said odor treating material into said receptacle;
closing said container with said odor treating material inside of said container by attaching said removably attachable lid to said receptacle;
dispensing said odor treating material directly onto an odor causing material.

9. The method for odor treatment according to claim 8, wherein said step of sealing said container further comprises the step of adding a safety seal onto said receptacle before said lid is attached to said receptacle.

10. The method for odor treatment according to claim 8, wherein said step of sealing said container further comprises the step of adding a safety seal onto said container after said lid is attached to said receptacle.

11. The method for odor treatment according to claim 8, wherein said odor treating material comprises 10-20% of activated carbon and 80-90% sodium bicarbonate.

12. The method for odor treatment according to claim 8, wherein when said odor treating material is dispensed from said container onto said odor causing material, said lid is about three inches or less from said odor causing material.

13. The method for odor treatment according to claim 8, wherein said lid comprises an open and closed position.

14. The method for odor treatment according to claim 8, further comprising the step of closing said container and storing said container for further use.

15. The method for odor treatment according to claim 14, further comprising the steps of:
providing a refill pack;
dispensing said odor treating material into said refill pack;
sealing said refill pack;
dispensing said odor treating material from said refill pack into said container.

16. A system for odor treatment comprising:
an odor treating material;
a container comprising a receptacle and a removably attachable lid;
wherein said removably attachable lid is removably attached to said receptacle by mating threads on said receptacle and said lid;
wherein said lid further comprises a base and a cover which each comprise at least one hole;
wherein said lid further comprises an open and a closed position;
wherein said open position is when said at least one hole in said cover is generally aligned with said at least one hole in said base and said closed position is when said holes are not generally aligned.

17. The system for odor treatment according to claim 16, wherein said odor treating material comprises at least two materials.

18. The system for odor treatment according to claim 17, wherein said odor treating material comprises 10-20% activated carbon and 80-90% sodium bicarbonate.

19. The system for odor treatment according to claim 16, wherein said system further comprises at least two containers, and wherein said first container is different from said second container.

20. The system for odor treatment according to claim 16, wherein said system further comprises at least two refill packs, and wherein said first refill pack is different from said second refill pack.
